(19) United States
(12) Patent Application Publication
(10) Pub. No.: US 2018/0228969 A1
(43) Pub. Date: Aug. 16, 2018

(54) Implantable Device and Methods for Reversing Overdose of a Substance

(71) Applicant: Ross MacDonald, New York, NY (US)

(72) Inventor: Ross MacDonald, New York, NY (US)

(21) Appl. No.: 15/430,974

(22) Filed: Feb. 13, 2017

(51) Int. Cl. A61M 5/172; A61M 5/142; A61B 5/00; A61B 5/11; A61B 5/08

(52) U.S. Cl. CPC A61M 5/1723; A61M 5/14276; A61B 5/4839; A61B 5/1118; A61B 5/0816; A61M 2230/63; A61M 2205/3523; A61B 5/0022; A61M 2230/42; A61M 2230/005; A61M 2205/04

(57) ABSTRACT

An implantable device for reversing an overdose of a substance in a person. The device measures the person's respiratory rate and/or the person's activity state and automatically injects a dose of overdose reversal agent in the person if the person's respiratory rate and/or the person's activity state indicate that the person may over overdosed on the substance. The device can administer subsequent doses if the person's respiratory rate and/or the person's activity state continue have not improved after the first dose. The device can include a wireless communication device to contact a third party after one or more doses have been administered.

### TECHNICAL FIELD

The present application relates generally to Implantable devices for reversing overdose of a substance in a person.

### BACKGROUND

In the United States there were more than 29,000 drug poisoning deaths attributable to opioids (heroin and opioid analgesics) in 2014, and the number of such deaths has increased steadily each year since 1999, when it stood at less than 6,000. This has led the United States Government Centers for Disease Control and Prevention to label overdose from prescription painkillers and heroin as an epidemic. Several responses to this dramatic increase in overdose death have been proposed and implemented including updated guidelines for the prescription of opioid analgesics, state-level prescription drug monitoring programs (PDMPs) to minimize “doctor-shopping” among opioid dependent patients, increasing use of medication assisted treatment (MAT) and other treatments for opioid dependence, and systems to train and distribute an opioid antagonist medication called naloxone to first responders.

Still, these efforts have thus far failed to stem the increase in overdose deaths and physicians are still left with limited options when treating patients who have experienced near-fatal overdose related to opioids, which is a common clinical situation, evidenced by the over 400,000 emergency department visits related to opioid analgesics or heroin in 2011. Treatment programs are often not readily available at the point of discharge from the hospital setting, limited by their availability, insurance coverage and often subject to wait lists. Even if treatment capacity were expanded to meet the need, many users are not prepared to engage in treatment subsequent to near-fatal overdose and remain at markedly elevated risk for death. Further, for patients who do go on to engage in treatment, such programs are not universally efficacious in reducing the risk of overdose death and elevated risk persists.

One effect caused by an opioid overdose is a depressed breathing or respiratory rate. Breathing in a human subject is effected by the expansion of the chest cavity (along with coordinated movement of the diaphragm) that generates a negative pressure within the thoracic cavity, thereby drawing in oxygenated ambient air and exchanging the biological waste product, carbon dioxide. The depressed respiratory rate is a result of direct action of the opioid agent on receptors in the individual's brain that control respiratory drive, such that the frequency of physical excursions of the chest cavity (i.e., breaths) slows down, preventing adequate influx of oxygenated air and efflux of waste products such as carbon dioxide. Either the lack of oxygen or the buildup of such waste products (acidosis) can lead to death of the individual by a critical lack of oxygen to the cells of the brain, heart or other vital organs or by arrhythmia of the heart, promoting the former.

One promising strategy that is being used increasingly is training third parties to recognize the signs of opioid overdose and to administer an opioid antagonist either via intranasal or subcutaneous injection. These third parties may be first responders such as police, paramedics, EMTs, and the like. The opioid overdose reversal agent achieves its effect by competitively removing the opioid agent from the receptors in the subject's brain, which are responsible for the control of respiratory drive.

Although training of third parties has increased, the problem remains that such third parties need to be contacted by someone to know about a potential overdose. Thus, when a person overdoses alone or together with other users, they are often left unconscious for prolonged periods of time, since no one is able to call for third-party assistance. This can be harmful to the overdosed user's health and can potentially be fatal. It would be desirable to overcome this and other problems in the art.

### SUMMARY

The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Other objects, advantages and novel features of the disclosure will be set forth in the following detailed description of the disclosure when considered in conjunction with the drawings.

One or more embodiments are directed to an implantable device for reversing an overdose of a substance in a person, the implantable device comprising a housing; a reservoir disposed in the housing to store an overdose reversal agent; an injector port in fluid communication with the reservoir, the injector port extending through the housing; a pressure source in fluid communication with the reservoir; an electromechanical gate disposed between the injector port and the reservoir, wherein the electromechanical gate has an open state that allows the overdose reversal agent to pass through the injector port, and a closed state that prevents the overdose reversal agent from passing through the injector port; a controller disposed in the housing, the controller in electrical communication with the electromechanical gate, the pressure source, and a thoracic cavity excursion sensor, wherein the controller determines a measured respiratory rate of the person using output signals from the thoracic cavity excursion sensor, and when the calculated respiratory rate is below a predetermined minimum respiratory rate for a predetermined rolling time period, the controller generates a first signal that causes the electromechanical gate to switch from the closed state to the open state, thereby administering a predetermined dose of the overdose reversal agent to the subcutaneous tissue of the individual through the injector port.

One or more embodiments are directed to a method for reversing an overdose of a substance using an implantable device in a person, the method comprising receiving data from a sensor disposed on or in the person, the data corresponding to a measured respiratory parameter of the person; determining whether the measured respiratory parameter is lower than a predetermined minimum respiratory parameter over a first rolling time period; when the measured respiratory parameter is lower than the predetermined minimum respiratory parameter over the first rolling time period administering a dose of an overdose reversal agent to the person from the implantable device; and automatically sending a notification message to a third party using a wireless communication device disposed in the implantable device.

One or more embodiments are directed to a method for reversing an overdose of a substance using an implantable device in a person, the method comprising receiving data from a sensor disposed on or in the person, the data corresponding to a measured respiratory rate of the person; receiving data from an accelerometer disposed in the implantable device, the data from the accelerometer corresponding to an active state or a rest state of the person; determining, using the data from the accelerometer, that the person is in the rest state for a predetermined time period; after determining that the person is in the rest state for the predetermined time period, determining whether the measured respiratory rate is lower than a predetermined minimum respiratory rate over a first rolling time period; when the measured respiratory rate is lower than the predetermined minimum respiratory rate over the first rolling time period administering a dose of an overdose reversal agent to the person from the implantable device; and automatically sending a notification message to a third party using a wireless communication device disposed in the implantable device.

### BRIEF DESCRIPTION OF THE DRAWINGS

For a fuller understanding of the nature and advantages of the present invention, reference is made to the following detailed description of preferred embodiments and in connection with the accompanying drawings, in which:

FIG. 1 is a block diagram of an implantable device for reversing overdose of a substance according to one or more embodiments;

FIG. 2 is a flow chart for a method of reversing a substance overdose in an individual using an implantable device according to one or more embodiments;

FIG. 3 is a flow chart for a method of reversing a substance overdose in an individual using an implantable device according to one or more embodiments; and

FIG. 4 is a flow chart 40 for a method of reversing a substance overdose.

### DETAILED DESCRIPTION

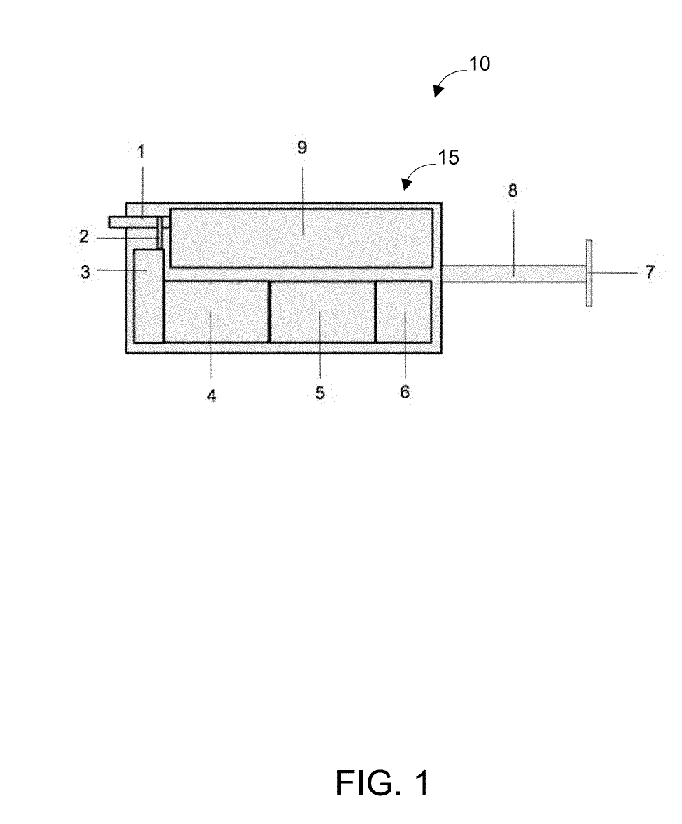

FIG. 1 is a block diagram of an implantable device 10 for reversing overdose of a substance according to one or more embodiments. The device 10 includes an injector port 1, an electromechanical gate 2, a controller 3, a battery 4, a wireless communications device 5, an accelerometer 6, a shear detector anchor 7, a shear detection arm 8, and an overdose reversal agent reservoir 9. The injector port 1 extends from the overdose reversal agent reservoir 9 to an outlet disposed outside of housing 15 of device 10.

The electromechanical gate 2 is disposed between the outlet of injector port 1 and the overdose reversal agent reservoir 9. The electromechanical gate 2 can be in an open position or a closed position. In the open position, the electromechanical gate 2 is adjusted (e.g., retracted, raised, etc.) to allow the overdose reversal agent to flow from the reservoir 9 to the outlet of injector port 1. Opening the electromechanical gate 2 causes a dose or volume (e.g., an aliquot) of overdose reversal agent to be administered to the individual through injector port 1. The length of time that the electromechanical gate 2 is open, in addition to the force applied by the optional pressure source, as described below, corresponds to the dose. In the closed position, the electromechanical gate 2 is adjusted so that it obstructs the flow of the overdose reversal agent. The electromechanical gate 2 can be a valve, a physical barrier, or other device that can selectively obstruct or allow the flow of overdose reversal agent, which generally is in liquid form.

The overdose reversal agent reservoir 9 holds a volume of an overdose reversal agent in liquid form, such as an overdose reversal agent for narcotics (e.g., opioids). An example of an overdose reversal agent is naloxone (e.g., NARCAN®). The overdose reversal agent reservoir 9 can hold the overdose reversal agent at a predetermined pressure, the predetermined pressure causing the overdose reversal agent to flow towards electromechanical gate 2 or injector port 1 (and into the individual) depending on whether electromechanical gate 2 is open or closed. The predetermined pressure in overdose reversal agent reservoir 9 can be maintained continuously in some embodiments, while in other embodiments the overdose reversal agent reservoir 9 is only pressurized when needed (e.g., on demand), such as just prior to administering a dose of the overdose reversal agent to the individual. The predetermined pressure can be obtained through a pressure source, such as a pump or other device in fluid communication with overdose reversal agent reservoir 9. Alternatively, overdose reversal agent reservoir 9 can be a flexible, squeezable bladder and the pressure source can be a bladder pump or other device (e.g., a pneumatic device) that can generate pressure or contract the flexible walls of the bladder. In some embodiments, a pressure sensor is in fluid communication with the overdose reversal agent reservoir 9 to sense the pressure generated by the pressure source. The pressure source and/or the pressure sensor can be in electrical communication (e.g., via a wired or a wireless connection) with controller 3.

After the device 10 is implanted in the individual, overdose reversal agent reservoir 9 can be re-filled in an outpatient procedure, for example by replacing the reservoir 9 or by a sterile injection of the overdose reversal agent directly into the reservoir 9 (i.e., while the device 10 remains implanted). The act of replacing the reservoir 9 can be similar to changing a battery in an implantable cardiac defibrillator device. In some embodiments, the dose administered to the individual is 0.4 to 1.0 mg of naloxone, or any value or range there between.

The controller 3 is in electrical communication with the electromechanical gate 2, the wireless communications device 5, the accelerometer 6, and the battery 4 (as a power source). As discussed in more detail herein, the controller 3 can send a control signal to the electromechanical gate 2 to cause it to open when the individual may have overdosed on a substance, such as a narcotic. To determine whether to send such a control signal, the controller 3 uses data provided by a thoracic cavity excursion sensor and/or the accelerometer 6.

The thoracic cavity excursion sensor is placed proximal to the individual's thoracic cavity (e.g., proximal to the individual's chest) to measure the respiratory rate of the individual. The controller 3 receives data (e.g., output signals) from the cavity excursion sensor wirelessly (through wireless communications device 5) or through a direct wired connection. In some embodiments, the thoracic cavity excursion sensor is disposed on or in device 10. Using the received data, controller 3 calculates a measured respiratory rate of the individual. The controller 3 then determines whether the measured respiratory rate falls below a predetermined minimum respiratory rate for a predetermined period of time. If the measured respiratory rate falls below the predetermined minimum respiratory rate for the predetermined period of time, controller 3 causes (e.g., via a control signal) the electromechanical gate 2 to be in the open state to administer a dose of the overdose reversal agent to the individual. In addition, controller 3 can cause (e.g., via a second control signal) a pressure source to pressurize the overdose reversal agent reservoir 9 to a predetermined pressure, which can be confirmed with an optional pressure sensor (as an input to controller 3), prior to causing the electromechanical gate 2 to be in the open state. In addition, controller 3 can confirm that the overdose reversal agent reservoir 9 is pressurized to the predetermined pressure (e.g., via an input signal from an optional pressure sensor) if the pressure source continuously maintains the overdose reversal agent reservoir 9 at the predetermined pressure. The dose administered can be a function of the length of time that electromechanical gate 2 is in the open state and the pressure (e.g., the predetermined pressure) on the overdose reversal agent in the overdose reversal agent reservoir 9. If the measured respiratory rate is at or above the predetermined minimum respiratory rate for the predetermined period of time, controller 3 causes (e.g., via a control signal) the electromechanical gate 2 to be in the closed state (or remain closed) to prevent the overdose reversal agent from flowing into the individual.

The predetermined minimum respiratory rate can be selected so that it generally corresponds with the respiratory rate of an individual who has overdosed on a substance, e.g., a narcotic substance such as an opioid. In general, the respiratory rate of an overdosed individual is low compared to an individual who has not overdosed. For example, an adult who has overdosed would have a respiratory rate of about 7 breaths per minute or less while an adult who has not overdosed (and is at rest) has a respiratory rate of about 12 breaths per minute to about 20 breaths per minute. Thus, it can be seen that a respiratory rate of about 5 breaths per minute that is sustained over a predetermined time period can be used as a reasonable proxy for determining whether an individual has overdosed. In other words, the predetermined minimum respiratory rate can be selected to be in the range of about 7 breaths per minute or fewer. As used herein, “about” means plus or minus 10% of the relevant value.

In some embodiments, controller 3 also determines the length of time that the individual has a measured respiratory rate. The length of time can be used to confirm that the reduced respiratory rate is due to an overdose and not from another cause, such as the individual holding his breath, meditating, or engaging in another activity that may also cause a temporary reduction in respiratory rate. For example, controller 3 can determine whether the measured respiratory rate is below the predetermined minimum respiratory rate for longer than a first predetermined maximum time period (e.g., a rolling time period of 30 seconds to 2 minutes) or a second predetermined maximum time period (e.g., a rolling time period of 3-5 minutes). In some embodiments, the measured respiratory rate can be an average or median respiratory rate measured over a rolling time period/window (e.g., 30 seconds to 5 minutes), such as the first and/or second predetermined maximum time periods. If controller 3 determines that the measured respiratory rate has fallen below the predetermined minimum respiratory rate for more than the first or second predetermined maximum time period, controller 3 causes (e.g., via a control signal) the electromechanical gate 2 to be in the open state to administer a dose of the overdose reversal agent to the individual. Prior to causing the electromechanical gate 2 to be in the open state, controller 3 can also confirm (e.g., via a pressor sensor) that the overdose reversal agent reservoir 9 is at a predetermined pressure and/or activate a pressure source to pressurize overdose reversal agent reservoir 9 to the predetermined pressure. However, if controller 3 determines that the measured respiratory rate at or above the predetermined minimum respiratory rate for more than the first or second predetermined maximum time period, controller 3 causes the electromechanical gate 2 to remain closed to prevent the overdose reversal agent from flowing into the individual (e.g., due to gravity or pressure provided by a pressure source). It is noted that the measured respiratory rate used by controller 3 can also be the average or median measured respiratory rate over the relevant time period.

Device 10 also includes an optional shear detection anchor 7 and an optional shear detection arm 8. Shear detection anchor 7 and shear detection arm 8 can measure the mechanical forces of the excursion of the chest to directly monitor the respiratory rate of the individual. For example, shear detection anchor 7 and shear detection arm 8 can measure the physical pressure exerted on device 10 by the workings of the respiratory muscles of the individual and the physical excursion of the chest cavity associated with breathing. The output signals generated by shear detection anchor 7 and shear detection arm 8 generate data, such as a tracing, which can be analyzed by controller 3 to measure the individual's respiratory rate. Controller 3 can use the measured respiratory rate, as determined from shear detection anchor 7 and shear detection arm 8, as an input (e.g., to determine whether to administer a dose of overdose reversal agent) in the same or substantially the same as the measured respiratory rate, as determined from a thoracic cavity sensor. The measured respiratory rate can be used by controller 3 along with inputs from accelerometer 6 and/or a pulse oximeter in some embodiments to determine whether to administer a dose of overdose reversal agent.

In some embodiments, controller 3 receives, as an input, the output signals from a pulse oximeter. The pulse oximeter can be included as part of device 10 or it can be a separate component that is in electrical communication (e.g., a wired or a wireless connection) with device 10. The pulse oximeter can be used in addition to or in place of mechanical respiratory rate detection (e.g., shear detection anchor 7 and shear detection arm 8, and/or thoracic cavity sensor) to provide both respiratory rate and oxygen saturation information to controller 6. In general, the oxygen saturation of the blood of an overdosed individual is lower than the oxygen saturation of the blood of an individual who has not overdosed. For example, an overdosed individual can have an oxygen saturation of about 90% or lower while an individual who has not overdosed would have an oxygen saturation of 95% to 100%. Thus, it can be seen that an oxygen saturation of less than about 90% can be used as a reasonable proxy for determining whether an individual has overdosed. Controller 3 can cause a dose of overdose reversal agent to be administered to the individual in response to the oxygen saturation or in response to a combination of the oxygen saturation and other data inputs, such as the measured pulse (as measured by the pulse oximeter), the measured respiratory rate and/or the individual's activity level (as measured by accelerometer 6).

Controller 3 is also in electrical communication with accelerometer 6, which can sense the individual's movement. In general, an overdosed individual remains still or at rest for a prolonged time period because the overdose causes the individual to be in a depressed state of consciousness. Thus, the output of accelerometer 6 can be used as a “check” on the measured respiratory rate since it is unlikely that an overdosed individual would have a low respiratory rate (e.g., below the predetermined minimum respiratory rate) while in an active state (e.g., while walking, turning the body, or other active movement). It is even less likely that an overdosed individual would have a low respiratory rate (e.g., below the predetermined minimum respiratory rate) for a sustained time period (e.g., longer than the first predetermined maximum time period) while in an active state. In some embodiments controller 3 determines whether the individual is in a rest or inactive state for a third or a fourth predetermined maximum time period, which can be the same or different than the first or second predetermined maximum time period, respectively. In some embodiments, controller 3 determines whether the individual is in a rest or inactive state for a predetermined time period of up to about 7 minutes to about 10 minutes.

Alternatively, controller 3 first determines that the individual is in a rest or inactive state for a predetermined time period (e.g., as described above), based on the output of accelerometer 6. If the individual has been in a rest or inactive state for the predetermined time period, controller 3 then checks the individual's measured respiratory rate (e.g., as described above) and administers the overdose reversal agent if the individual's measured respiratory rate is low (e.g., below the predetermined minimum respiratory rate) for a sustained time period (e.g., longer than the first predetermined maximum time period). In this alternative embodiment, it can be seen that the output of accelerometer 6 can be used as a prerequisite or gate for whether controller 3 needs to check the individual's measured respiratory rate.

In some embodiments controller 3 administers a dose of overdose reversal agent to be administered to the individual in one or more of the following scenarios. As discussed above, administering a dose of overdose reversal agent includes opening electromechanical gate 2 and, optionally, activating a pressure source (e.g., prior to opening electromechanical gate 2) to increase the pressure in overdose reversal agent reservoir 9 to a predetermined pressure to thereby cause overdose reversal agent to flow through injector port 1 into the individual. As discussed, the pressure in overdose reversal agent reservoir 9 can also be checked (e.g., via a pressure sensor) prior to opening electromechanical gate 2.

A. Controller 3 determines that the measured respiratory rate of the individual falls below the predetermined minimum respiratory rate for any period of time;

B. Controller 3 determines that the measured respiratory rate of the individual falls below the predetermined minimum respiratory rate for longer than a first predetermined maximum time period or a second predetermined maximum time period, the second predetermined maximum time period greater than the first predetermined maximum time period;

C. Controller 3 determines that (1) the measured respiratory rate of the individual falls below the predetermined minimum respiratory rate for any period of time and (2) the individual is currently in an inactive state (i.e., the output of accelerometer 6 indicates that the individual is not moving);

D. Controller 3 determines that (1) the measured respiratory rate of the individual falls below the predetermined minimum respiratory rate for any period of time and (2) the individual has been in an inactive state for longer than a third predetermined maximum time period or a fourth predetermined maximum time period, the fourth predetermined maximum time period greater than the third predetermined maximum time period;

E. Controller 3 determines that (1) the measured respiratory rate of the individual falls below the predetermined minimum respiratory rate for longer than the first or second predetermined maximum time period and (2) the individual is currently in an inactive state;

F. Controller 3 determines that (1) the measured respiratory rate of the individual falls below the predetermined minimum respiratory rate for longer than the first or second predetermined maximum time period and (2) the individual has been in an inactive state for longer than the third or fourth predetermined maximum time period;

G. Controller 3 first determines that the individual has been in an inactive state for longer than the third or fourth predetermined maximum time period. If so, controller 3 then determines that the measured respiratory rate of the individual falls below the predetermined minimum respiratory rate for any period of time;

H. Controller 3 first determines that the individual has been in an inactive state for longer than the third or fourth predetermined maximum time period. If so, controller 3 then determines that the measured respiratory rate of the individual falls below the predetermined minimum respiratory rate for longer than the first or second predetermined maximum time period;

I. Controller 3 determines that (1) the individual has been in an inactive state for longer than the third or fourth predetermined maximum time period and (2) the oxygen saturation of the individual is below a predetermined minimum oxygen saturation (e.g., 90%);

J. Controller 3 determines that (1) the oxygen saturation of the individual is below the predetermined minimum oxygen saturation and (2) the measured respiratory rate of the individual falls below the predetermined minimum respiratory rate for longer than the first or second predetermined maximum time period;

K. Controller 3 determines that (1) the oxygen saturation of the individual is below the predetermined minimum oxygen saturation, (2) the measured respiratory rate of the individual falls below the predetermined minimum respiratory rate for longer than the first or second predetermined maximum time period, and (3) the individual has been in an inactive state for longer than the third or fourth predetermined maximum time period; or

L. Controller 3 determines that (1) the oxygen saturation of the individual is below the predetermined minimum oxygen saturation and (2) the measured respiratory rate of the individual falls below the predetermined minimum respiratory rate for longer than the first or second predetermined maximum time period; and (3) the pulse of the individual falls below a predetermined minimum pulse.

The converse of the foregoing scenarios is also true. For example, when controller 3 determines that the measured respiratory rate (as measured by the cavity excursion sensor) and/or the individual's state of activity (as measured by accelerometer 6) indicate that the individual has not overdosed, controller 3 prevents (via a control signal to close electromechanical gate 2) the overdose reversal agent from being administered to the individual. Alternatively, controller 3 does not need to send any control signals to prevent the overdose reversal agent from being administered to the individual since the default position of electromechanical gate 2 can be set to the closed state.

After a dose of the overdose reversal agent has been administered (e.g., in the case of one or more of scenarios A-L, above), controller 3 returns to monitoring the individual's respiratory rate and/or activity level. If controller 3 determines that one or more of scenarios A-L exists after the overdose reversal agent has been administered, controller 3 can administer a second dose of the overdose reversal agent. This process can continue until one of the following occurs: (a) the individual's physical conditions have improved (i.e., the measured respiratory rate rises above the predetermined minimum respiratory rate and/or activity (movement) of the individual is detected by accelerometer 6); (b) a predetermined maximum number of doses of the overdose reversal agent have been administered; or (c) the device 10 is deactivated, for example by first responders or medical personnel.

In some embodiments, controller 3 waits for a predetermined time period after administering a dose of the overdose reversal agent before re-checking the individual's physical conditions to determine if a second dose is needed (e.g., if one or more of scenarios A-L exists). This predetermined time period can be set to allow adequate time for the overdose reversal agent to take effect in the individual. In some embodiments, this time period is from about 2 minutes to about 10 minutes, about 4 minutes, about 6 minutes, about 8 minutes, or any value or range between any two of the foregoing values.

In some embodiments, controller 3 administers a first dose when it determines that a first scenario (e.g., scenario A) exists and then administers a second dose when it determines that a second scenario (e.g., scenario B) exists, and so on. As discussed, controller 3 can wait for a time period between administering the first dose and determining whether the individual needs a second dose. The order of the scenarios in the foregoing example can be selected in an increasing order of “sensitivity.” That is, the first dose can be administered in response to scenario A (measured respiratory rate less than predetermined minimum respiratory rate for any period of time) while the second dose is only administered if one of the “checks” passes. For example, the second dose can be administered only in response to scenario B, which requires a sustained low respiratory rate. In another example, the first dose is administered in response to scenario B when the low respiratory rate is sustained for a first predetermined maximum time period and the second dose is administered in response to scenario B when the low respiratory rate is sustained for a second predetermined maximum time period, the second predetermined maximum time period being longer than the first predetermined maximum time period. In some embodiments, the first predetermined time period is from 30 seconds to 2 minutes and the second predetermined time period is from 3 minutes to 5 minutes.

In another example, the second dose can be administered only in response to scenario D, which requires input from accelerometer 6 in addition to the sustained low respiratory rate. Of course, the foregoing are examples and other combinations of dosing and scenarios are within the scope of these embodiments, including for three or more doses. In addition or in the alternative, controller 3 can increase dosing from the first dose (e.g., 0.4 mg of naloxone) to the second dose (e.g., 0.6 mg of naloxone), and/or from the second dose (e.g., 0.6 mg of naloxone) to the third dose (e.g., 0.8 mg of naloxone).

When controller 3 determines that it is appropriate to administer a dose of the overdose reversal agent, controller 3 can automatically generate a notification signal or message to notify an appropriate party (e.g., first responders, etc.) that the individual may have overdosed on a substance. The notification signal can be transmitted to the appropriate party via wireless communications center 5, which can include a cellular or WiFi-enabled communications device. In some embodiments, device 10 includes a location-sensitive device, such as a GPS device comprising GPS circuitry and instructions, for determining the location of the device (and thus the overdosed individual). The notification signal can include the location (e.g., GPS coordinates, street address, etc.) of the device, which can assist the receiving party (or a third party notified by the receiving party) in locating the overdosed individual. The notification signal can also include the identity of or a unique identifier of the overdosed individual. For example, the notification signal can include the individual's name and date of birth. In addition or in the alternative, the notification signal can include a unique identifier assigned to the overdosed individual. An example of such a unique identifier can include the serial number of the device 10, the individual's social security number, an encrypted or hashed version of either of these identifiers, or other identifier associated with individual, any of which can be stored in a database accessible by the receiving party.

The wireless communications center 5 can also send data collected by the device 10. The data can include the measured respiratory rate, the output data of accelerometer 6, and/or the time that any doses have been administered. In some embodiments, the device 10 has an internal memory unit that can store the data collected by the thoracic cavity excursion sensor and the accelerometer 6. The data stored on the internal memory unit can be limited (e.g., to the size of the memory until) to a rolling time window (e.g., past 12 hours). The data stored on the internal memory unit can also include the time that the data was collected and the time and amount of any doses administer to the individual. Some or all of this data can be sent in conjunction with the notification signal to the receiving party using wireless communications center 5. In addition or in the alternative, some or all of the data can be sent to a device held by a first responder or other party who is physically close to the individual, for example over a local wireless connection such as Bluetooth. Data sent from the device 10 using wireless communications center 5 can also be used to monitor the individual or to debug the device 10. Also, device 10 can receive data over wireless communications center 5 to program or re-program the device 10, such as the instructions and parameters used by controller 3.

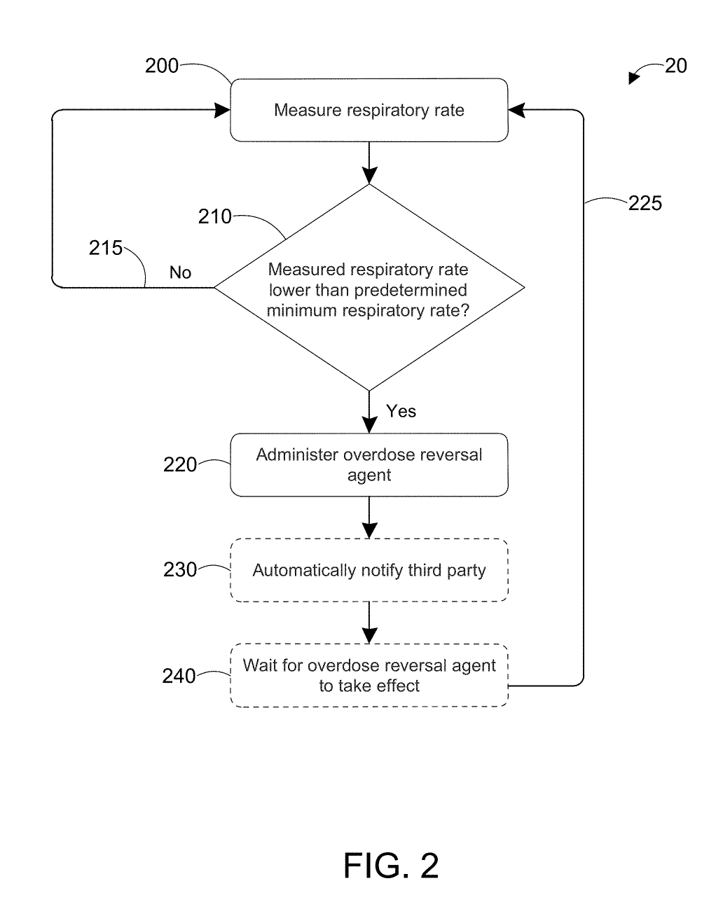

FIG. 2 is a flow chart 20 for a method of reversing a substance overdose in an individual using an implantable device (e.g., device 10 described above) according to one or more embodiments. In step 200, the device measures the individual's respiratory rate, for example by using data or signals output from a thoracic cavity excursion sensor. As discussed above, the thoracic cavity excursion monitor can be external or internal to the device, and can be electrically coupled to the device with a wired or a wireless connection. In step 210, the device determines whether the measured respiratory rate is lower than a predetermined minimum respiratory rate. As discussed above, the predetermined minimum respiratory rate can be selected so that it corresponds with the typical respiratory rate of an overdosed individual, such as about 7 breaths per minute to about 10 breaths per minute. However, the present concepts can be extended to other breath rates, e.g., 5-7 breaths per minute, or another rate. In addition or in the alternative, the determination in step 210 can be based on the average or median measured respiratory rate over a predetermined rolling time period (e.g., over the past 30 seconds to over the past 5 minutes).

If the measured respiratory rate is greater than or equal to the predetermined minimum respiratory rate at 210, flow chart 20 loops back at 215 to step 200 to re-measure the respiratory rate. This loop continues until the device determines that the measured respiratory rate is lower than a predetermined minimum respiratory rate at 210 (e.g., over a predetermined rolling time period as discussed above), in which case flow chart 20 proceeds to step 220. In step 220, the device administers a dose of overdose reversal agent to the individual through an external port on the device. After the dose is administered, the flow chart at 225 loops back to step 200 to re-measure the respiratory rate.

In an alternative embodiment, flow chart 20 proceeds to optional step 230 and/or optional step 240. In optional step 230, the device automatically sends a notification (e.g., via a wireless communications module in the device over a cellular or WiFi network) to alert a third party that the individual has overdosed. The notification can include identifying information for the individual and the individual's location (e.g., if the device includes a GPS unit). In some embodiments, the third party is selected based, at least in part, on the individual's location. For example, the third party can be a first responder located in the same town or region as the individual.

The flow chart can also proceed to step 240, in which the device waits for a waiting time period to allow the overdose reversal agent to take effect in the individual. The waiting time period in step 240 can be 2 minutes to 10 minutes, or any value or range there between. After the waiting time period in step 240 is complete, the flow chart loops back at 225 to step 200, as discussed above.

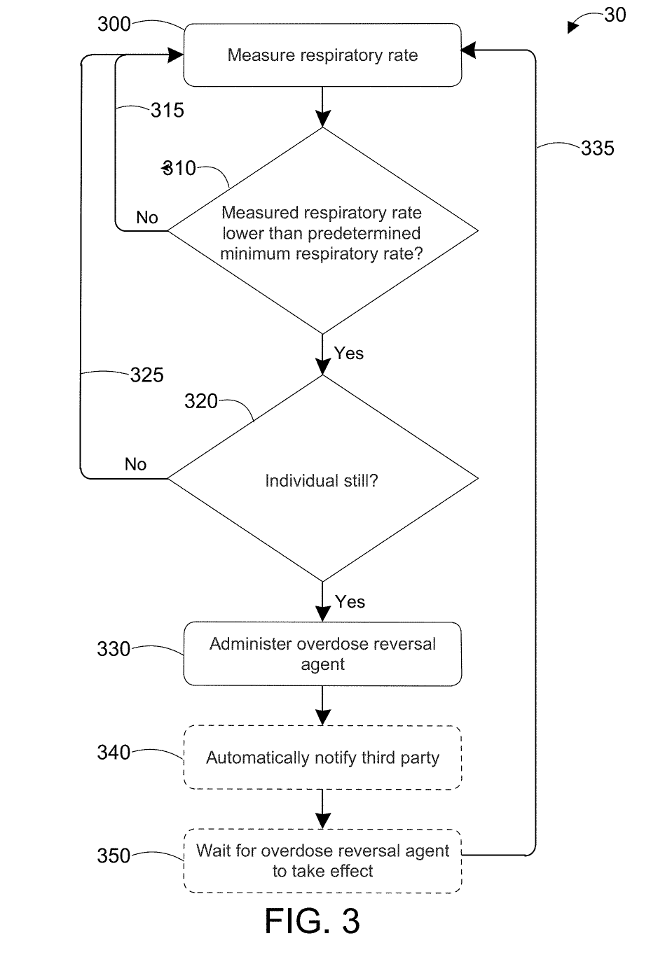

FIG. 3 is a flow chart 30 for a method of reversing a substance overdose in an individual using an implantable device (e.g., device 10 described above) according to one or more embodiments. In step 300, the device measures the individual's respiratory rate, as discussed above. In step 310, the device determines whether the measured respiratory rate is lower than a predetermined minimum respiratory rate. As discussed above, this determination can be based on the mean or median measured respiratory rate over a predetermined rolling time period (e.g., over the past 30 seconds to over the past 5 minutes). If the measured respiratory rate is greater than or equal to the predetermined minimum respiratory rate (e.g., over a predetermined rolling time period as discussed above), flow chart 30 loops back at 315 to step 300, similar to flow chart 20. However, if the measured respiratory rate is lower than the predetermined minimum respiratory rate at 310, flow chart 30 proceeds to step 320.

In step 320, the device uses the output of an accelerometer or other motion-sensing device to determine if the individual is still or at rest. The device can determine if the individual is still (or not) over a rolling time period (e.g., over the past 30 seconds to over the past 5 minutes). If the device determines that the individual is not still (i.e., is currently moving or has moved during the rolling time period), the flow chart at 325 loops back to step 300 to re-measure the individual's respiratory rate. If the device determines that the individual is still or at rest, (i.e., is currently still or has not moved during the rolling time period), the flow chart proceeds to 330 to administer a dose of overdose reversal agent to the individual through an external port on the device. After the dose is administered, the flow chart at 335 loops back to step 300 to re-measure the respiratory rate. The accelerometer or motion-sensor may be used as a first or primary detector, with the breathing rate detector being used as a secondary detector in some embodiments.

In an alternative embodiment, flow chart 30 proceeds to optional step 340 and/or optional step 350. In optional step 340, the device automatically sends a notification (e.g., via a wireless communications module in the device over a cellular or WiFi network) to alert a third party that the individual has overdosed, as discussed above in step 230. The notification can include identifying information for the individual and the individual's location (e.g., if the device includes a GPS unit). In some embodiments, the third party is selected based, at least in part, on the individual's location. For example, the third party can be a first responder located in the same town or region as the individual.

In optional step 350, the device waits for a waiting time period to allow the overdose reversal agent to take effect in the individual, as discussed above in step 240. After flow chart 30 proceeds through optional step 340 and/or optional step 350, it loops back in 335 to step 300.

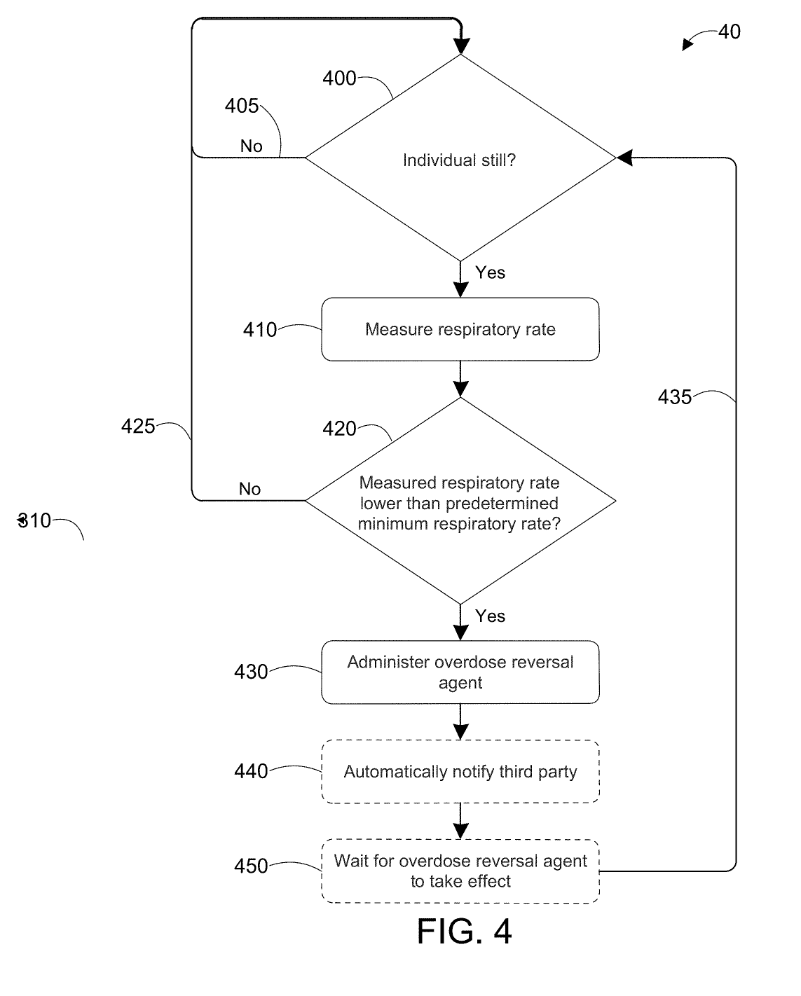

FIG. 4 is a flow chart 40 for a method of reversing a substance overdose in an individual using an implantable device (e.g., device 10 described above) according to one or more embodiments. In step 400, the device uses the output of an accelerometer or other motion-sensing device to determine if the individual is still or at rest, for example over a rolling time period (e.g., over the past 30 seconds to over the past 5 minutes). Step 400 can be the same or substantially the same as step 320, described above. If the device determines that the individual is not still or at rest (i.e., is currently moving or has moved during the rolling time period), flow chart 40 loops back at 405 to step 400 re-check whether the individual is still or at rest. In some embodiments, the device can wait for a predetermine time period (e.g., about 1 minute to about 10 minutes) prior to rechecking whether the individual is still or at rest.

If the individual is still or at rest (e.g., over the rolling time period), the device measures the individual's respiratory rate in step 410. In step 420, the device determines whether the measured respiratory rate is lower than a predetermined minimum respiratory rate. Steps 410 and 420 can be the same or substantially the same as steps 300 and 310, respectively. If the measured respiratory rate is not lower than the predetermined minimum respiratory rate, flow chart 40 loops back at 425 to step 400 to re-check whether the individual is still or at rest. In some embodiments, the device can wait for a predetermine time period (e.g., about 1 minute to about 10 minutes) prior to rechecking whether the individual is still or at rest. If the measured respiratory rate is lower than the predetermined minimum respiratory rate, the device administers a dose of overdose reversal agent to the individual through an external port on the device. After the dose is administered, the flow chart at 435 loops back to step 400 to re-check whether the individual is still or at rest.

In an alternative embodiment, flow chart 40 proceeds to optional step 440 and/or optional step 450, which are the same or substantially the same as optional steps 340 and 350, respectively, described above.

Those skilled in the art will appreciate the many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The present invention should therefore not be considered limited to the particular embodiments described above. Various modifications, equivalent processes, as well as numerous structures to which the present invention may be applicable, will be readily apparent to those skilled in the art to which the present invention is directed upon review of the present disclosure.

What is claimed is:

1. An implantable device for reversing an overdose of a substance in a person, the implantable device comprising:
a housing;
a reservoir disposed in the housing to store an overdose reversal agent;
an injector port in fluid communication with the reservoir, the injector port extending through the housing;
a pressure source in fluid communication with the reservoir;
an electromechanical gate disposed between the injector port and the reservoir, wherein the electromechanical gate has an open state that allows the overdose reversal agent to pass through the injector port, and a closed state that prevents the overdose reversal agent from passing through the injector port;
a controller disposed in the housing, the controller in electrical communication with the electromechanical gate, the pressure source, and a thoracic cavity excursion sensor, wherein:
the controller determines a measured respiratory rate of the person using output signals from the thoracic cavity excursion sensor, and
when the calculated respiratory rate is below a predetermined minimum respiratory rate for a predetermined rolling time period, the controller generates a first signal that causes the electromechanical gate to switch from the closed state to the open state, thereby administering a predetermined dose of the overdose reversal agent to the subcutaneous tissue of the individual through the injector port.

2. The implantable device of claim 1, further comprising an accelerometer disposed in the housing, the accelerometer in electrical communication with the controller.

3. The implantable device of claim 2, wherein the controller determines whether the person is in an active state or a rest state using output signals from the accelerometer.

4. The implantable device of claim 3, wherein the controller causes the electromechanical gate to switch from the closed state to the open state only when (a) the calculated respiratory rate is below the predetermined minimum respiratory rate for the predetermined rolling time period and (b) the person is in the rest state.

5. The implantable device of claim 4, wherein the controller causes the electromechanical gate to switch from the closed state to the open state only when the person is in the rest state for a second predetermined rolling time period.

6. The implantable device of claim 1, further comprising a wireless communications device disposed in the housing, the wireless communication device in electrical communication with the controller.

7. The implantable device of claim 6, wherein the controller sends a notification message to a third party using the wireless communications device when the controller causes the electromechanical gate to switch from the closed state to the open state.

8. The implantable device of claim 7, wherein the wireless communications device comprises a cellular communications device.

9. The implantable device of claim 6, further comprising a GPS device disposed in the housing, and wherein the notification message includes a location of the GPS device.

10. The implantable device of claim 1, said injector port further being coupled to a pump.

11. A method for reversing an overdose of a substance using an implantable device in a person, the method comprising:
receiving data from a sensor disposed on or in the person, the data corresponding to a measured respiratory parameter of the person;
determining whether the measured respiratory parameter is lower than a predetermined minimum respiratory parameter over a first rolling time period;
when the measured respiratory parameter is lower than the predetermined minimum respiratory parameter over the first rolling time period:
administering a dose of an overdose reversal agent to the person from the implantable device; and
automatically sending a notification message to a third party using a wireless communication device disposed in the implantable device.

12. The method of claim 11, further comprising:
determining an activity state of the person using an accelerometer disposed in the implantable device, the activity state comprising an active state or a rest state; and
administering the dose of the overdose reversal agent to the person only when (a) the measured respiratory parameter is lower than the predetermined minimum respiratory parameter over the first rolling time period and (b) the person is in the rest state.

13. The method of claim 12, wherein the rest state is determined over a second rolling time period.

14. The method of claim 11, further comprising:
determining a location of the implantable device using a GPS device disposed in the implantable device; and
wherein the notification message includes the location of the implantable device.

15. The method of claim 11, said respiratory parameter comprising any of a breathing rate or an oxygen level in said person's blood.

16. The method of claim 12, further comprising:
after administering the first dose of the overdose reversal agent to the person,
receiving second data from the sensor disposed on or in the person, the second data corresponding to a second measured respiratory rate of the person;
determining whether the second measured respiratory rate is lower than the predetermined minimum respiratory rate over a second rolling time period; and
when the measured respiratory rate is lower than the predetermined minimum respiratory rate over the second rolling time period, administering a second dose of the overdose reversal agent to the person from the implantable device.

17. The method of claim 16, further comprising:
after administering the first dose of the overdose reversal agent to the person, waiting for a wait time period prior to determining whether the second measured respiratory rate is lower than the predetermined minimum respiratory rate over the second rolling time period.

18. The method of claim 16, wherein the second rolling time period is greater than the first rolling time period.

19. The method of claim 16, wherein the second dose is higher than the first dose.

20. The method of claim 16, further comprising:
after administering the first dose of the overdose reversal agent to the person,
determining a second activity state of the person using the accelerometer, the second activity state comprising an active state or a rest state; and
administering the second dose of the overdose reversal agent to the person only when (a) the second measured respiratory rate is lower than the predetermined minimum respiratory rate over the second rolling time period and (b) the second activity state of the person is the rest state.

21. The method of claim 11, further comprising:
receiving data from a second sensor disposed on or in the person, the data corresponding to a measured oxygen saturation of the person's blood; and
administering the dose of the overdose reversal agent to the person from the implantable device only when (a) the measured respiratory rate is lower than the predetermined minimum respiratory rate over the first rolling time period and (b) the measured oxygen saturation of the person's blood is lower than a predetermined minimum oxygen saturation.

22. A method for reversing an overdose of a substance using an implantable device in a person, the method comprising:
receiving data from a sensor disposed on or in the person, the data corresponding to a measured respiratory rate of the person;
receiving data from an accelerometer disposed in the implantable device, the data from the accelerometer corresponding to an active state or a rest state of the person;
determining, using the data from the accelerometer, that the person is in the rest state for a predetermined time period;
after determining that the person is in the rest state for the predetermined time period, determining whether the measured respiratory rate is lower than a predetermined minimum respiratory rate over a first rolling time period;
when the measured respiratory rate is lower than the predetermined minimum respiratory rate over the first rolling time period:
administering a dose of an overdose reversal agent to the person from the implantable device; and
automatically sending a notification message to a third party using a wireless communication device disposed in the implantable device.

23. The method of claim 22, further comprising:
receiving data from a second sensor disposed on or in the person, the data corresponding to a measured oxygen saturation of the person's blood; and
administering the dose of the overdose reversal agent to the person from the implantable device only when (a) the measured respiratory rate is lower than the predetermined minimum respiratory rate over the first rolling time period, (b) the measured oxygen saturation of the person's blood is lower than a predetermined minimum oxygen saturation, and (c) the person is in the rest state for the predetermined time period.
